(19) United States
(12) Patent Application Publication
(10) Pub. No.: US 2011/0200683 A1
(43) Pub. Date: Aug. 18, 2011

(54) Hydrogen peroxide solution producing first aid substance, packaging, and treatment

(75) Inventor: Samuel David Piper, Carmichael, CA (US)

(73) Assignee: Piper Medical, Inc, Carmichael, CA (US)

(21) Appl. No.: 12/932,029

(22) Filed: Feb. 15, 2011

Related U.S. Application Data
(60) Provisional application No. 61/338,216, filed on Feb. 16, 2010.

(51) Int. Cl. A61K 33/40; A61P 17/02; A61P 31/02; A61J 1/00; A61J 3/02

(52) U.S. Cl. 424/616; 604/408; 206/459.5

(57) ABSTRACT

The present invention is directed generally to a first aid treatment package the contents of which can be mixed with available water to create a solution useful for the treatment of wounds, and more specifically, a first aid treatment package the contents of which include a adduct of hydrogen peroxide which when mixed with available water create a hydrogen peroxide solution for the first aid treatment of wounds in remote locations or under circumstances when normal community resources are unavailable.

### CROSS-REFERENCE TO RELATED APPLICATIONS

This application claims the benefit under 35 U.S.C. 119(e) of U.S. provisional application Ser. No. 61/338,216 filed Feb. 16, 2010, which is incorporated by reference herein in its entirety

### STATEMENT REGARDING FEDERALLY SPONSORED RESEARCH

Not Applicable

### BACKGROUND OF THE INVENTION

1. Field of the Invention

This invention pertains generally to an antimicrobial substance for treatments of skin abrasions, minor cuts, and major open wounds that are in needed of cleaning and in danger of infection, and more particularly for treatment of such injuries in remote locations or in situations where a light weight low mass alternative would be preferable.

2. Description of the Background Art

A number of methods commonly exist for cleaning of injuries that involve a break in the skin and for cleaning such injuries of foreign material and potentially dangerous antigens. Depending on the severity of the injury, various approaches are used including: soap and water; saline; hydrogen peroxide solution; isopropyl alcohol; and other liquid antimicrobial compounds. Liquid antimicrobial solutions can be particularly effective because they provide both antimicrobial action against antigens present and also provide mechanical means for removing antigens and debris from the wound.

The care of significant wounds in the wilderness, remote locations, or in situations where access to normal supplies has been interrupted by a catastrophic community event presents a great challenge. Often people participating in remote and/or outdoor activities (i.e. backpacking, sailing, world travel, bicycling, mountain biking, endurance running, cross country skiing, snow shoeing, kayaking, mountain climbing, rock climbing, flying, . . . ) are significantly impacted by constraints on how much weight they can carry or take with them. Although the lack of access to sterile supplies and hospital care renders useless or impossible many standard treatments that would be available for routine wound care in normal functioning community settings, the likelihood of a serious injury is small enough that many choose, due to the encumbrance of additional weight, to not carry the necessary medical supplies that may be needed for a serious injury while traveling or participating in activities that occur in remote locations. Although many medical supplies are unavailable for people in remote locations, often water is available.

Therefore the need exists for a light weight compact easy to carry product or method by which significant antimicrobial and mechanical wound cleaning and the promotion of healing can be realized once the product has been re-constituted with water.

### BRIEF SUMMARY OF THE INVENTION

The present invention generally comprises the use of a dry substance, when combined with non-sterile water, will produce a hydrogen peroxide solution of equivalent strength, or stronger, as is commonly available in liquid form in pharmacies and drug retail outlets around the world. Such a solution is useful for killing antigens present in the water, killing antigens present in a wound, rapidly stopping capillary bleeding, and cleaning a wound of any foreign material. The present invention is also useful in the soaking and cleaning of any clothe (likely to be harboring antigens as well) prior to using as a cover for a wound.

Hydrogen peroxide solution available in most pharmacies is normally a 3% or 6% solution consisting almost entirely of water. At normal atmospheric temperatures and pressures, pure (i.e. 100%) hydrogen peroxide is a liquid form, is relatively unstable, and dangerous to deal with, and therefore problematic for storage and carrying on ones person. The present invention comprises using an adduct (or salt) of hydrogen peroxide with sufficient solubility to allow easy preparation of an aqueous hydrogen peroxide solution of a minimum necessary concentration to fulfill it's intended function. There are a number of hydrogen peroxide adducts available with varying solubilities and stability profiles. These adducts are stable (provided they are kept dry), occupy little volume, are light weight, and easy and safe to handle. When mixed with water, of varying sterility and salinity, these adducts are capable of providing usable liquid with sufficient hyrdrogen peroxide present to kill antigens present in the water and wound, and thus mitigate the risk of infection and speed the rate of healing. The use of the present invention has all the advantages of a normal hydrogen peroxide solutions with the added value of being light weight, compact, easy to carry, stable, and can be potentially mixed to a stronger concentration if needed or desired.

Although not intended to be limiting in scope or form, the preferred embodiment of the current invention includes packaging pre-measured hydrogen peroxide adducts in sealed foil/polymer pouches. When, and if needed, the foil/polymer pouch may be torn open and poured into a known and specified amount of water of varying sterility and salinity. Once gently shaken and allowed to sit for a minute, many of the antigens present in the water will be killed and the liquid will be ready for treating the wound in exactly the same means and with the same effect as if provided with USP grade hydrogen peroxide (although the concentration may be varied if desired).

Foil/polymer pouches have a long history of demonstrated packaging stability and are a very light weight and safe means for carrying adducts of hydrogen peroxide. Foil/polymer pouches are welded, sealed, or formed into closed pouches with sides consisting of laminated sheets of materials. A laminate sheet material consisting of an outer layer of polyester, core layer of aluminum foil, and an inside layer of polyethylene has been demonstrated to be more than adequate for the purpose, although durability of the packaging configuration can be increased by including a layer of nylon between the foil and the polyethylene. Key to the success of the packaging is the vapor and gas impermeability of aluminum foil combined with the inert properties of polyethylene. The polyester is primarily used for it toughness as an outer layer to protect against inadvertent puncturing or opening of the foil/polymer pouch. Often the pouch is equipped with one or more pre-cut notches or slits to facilitate tearing open the pouch when needed. Instructions can be included with, internally or externally, or printed on the pouch for easy reference.

An object of the invention is to provide a means to create a solution for treating wounds in remote or disaster situations.

Another object of the invention is to provide a light weight and easy to carry means of providing a solution for treating wounds in remote or disaster situations.

Another object of the invention is to combine a light weight and easy to carry product with commonly available water of variable sterility and salinity to create a solution for treating wounds in remote or disaster situations.

Another object of the invention is to combine a light weight and easy to carry product with commonly available water to create a hydrogen peroxide solution for treating wounds in remote or disaster situations.

Another object of the invention is to combine a light weight and easy to carry product with commonly available non-sterile water to create a solution capable of killing antigens present in wounds in remote or disaster situations.

Another object of the invention is to combine a light weight and easy to carry product with commonly available water to create a solution capable of rapidly stopping capillary bleeding of wounds in remote or disaster situations.

Another object of the invention is to combine a light weight and easy to carry product with commonly available water to create a solution for cleaning wounds of foreign material in remote or disaster situations.

Another object of the invention is to provide the means and method to facilitate cleaning of wounds in remote or disaster situations.

Another object of the invention is to provide the means and method to promote faster healing of wounds in remote or disaster situations.

Further objects and advantages of the invention will be brought out in the following portions of the specification, wherein the detailed description is for the purpose of fully disclosing preferred embodiments of the invention without placing limitations thereon.

### BRIEF DESCRIPTION OF THE DRAWINGS

The invention will be more fully understood by reference to the following drawings which are for illustrative purposes only:

FIG. 1 is a front view of the invention indicating the presence of an adduct of hydrogen peroxide present inside the packaging pouch prior to making the final seal during the filling and fabrication process.

FIG. 2 is a perspective view of the invention indicating the presence of an adduct of hydrogen peroxide present inside the packaging pouch prior to making the final seal during the filling and fabrication process.

FIG. 3 is a front view of the invention indicating the presence of an adduct of hydrogen peroxide present inside the fully sealed packaging pouch in its final ready to carry and use when needed configuration.

### LIST OF REFERENCE NUMERALS

1 Hydrogen Peroxide Treatment Pack
2 Foil Pouch
3 Hydrogen Peroxide Adduct
4 First Opening Slit
5 Second Opening Slit
6 Pre-Fill Weld
7 Post-Fill Weld
8 First Laminated Sheet
9 Second Laminated Sheet

### DETAILED DESCRIPTION OF THE INVENTION

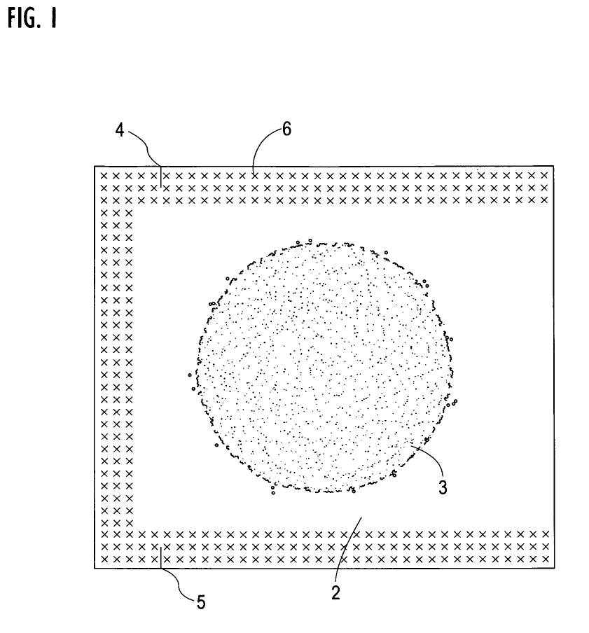

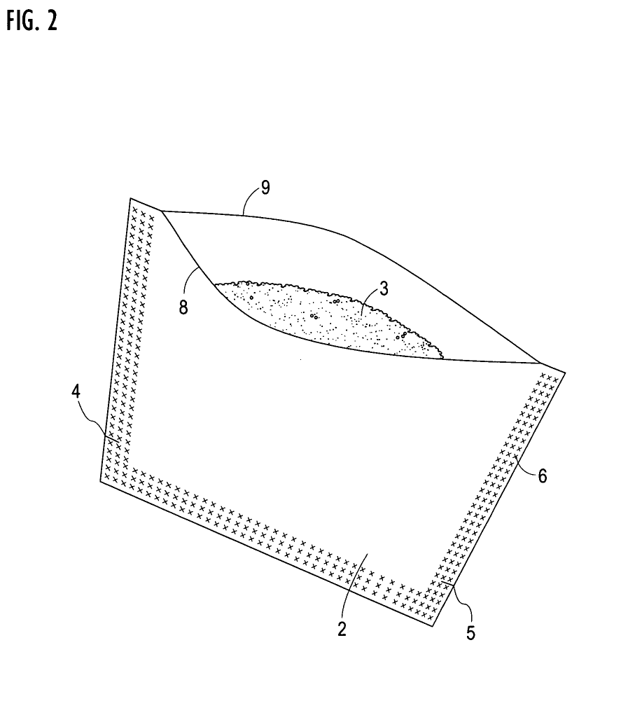

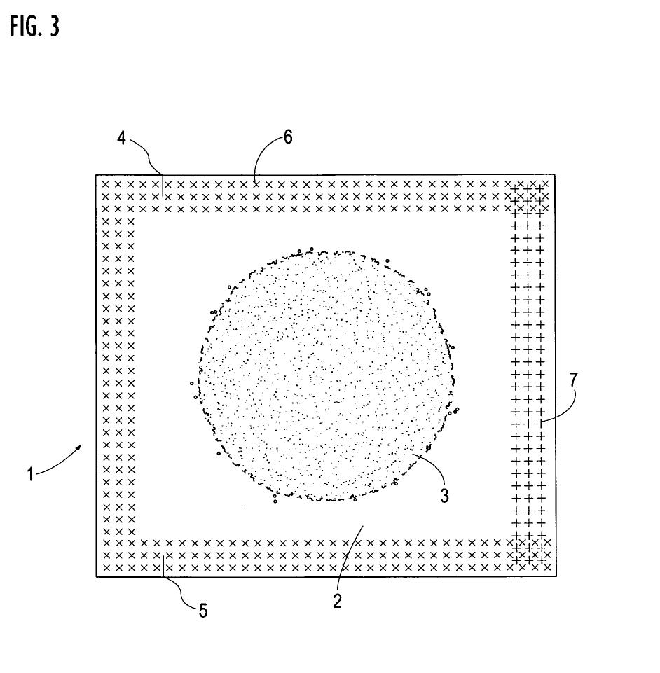

Referring more specifically to the drawing, for illustrative purposes the present invention is embodied in the apparatus generally shown in FIGS. 1, 2, and 3. It will be appreciated that the apparatus may vary as to configuration and as to details in the parts without departing from the basic concepts as disclosed herein.

Referring first to FIGS. 1, 2, and 3 the invention is generally indicated by hydrogen peroxide treatment pack 1. The fabrication of hydrogen peroxide treatment pack 1 generally consists of starting with two laminated sheets, first laminated sheet 8 and second laminated sheet 9, that are oriented so that their polyethylene exposed layers face each other, and are pre-sealed around the perimeter on three sides by a pre-fill weld 6 thus forming foil pouch 2. Hydrogen peroxide adduct 3 is then placed inside foil pouch 2, placed in a vacuum chamber, and sealed on the remaining open side by post-fill weld 7. Opening slits, as indicated by first opening slit 4 and second opening slit 5, may be added before or after filling of foil pouch 2, and are used to facilitate tearing open of hydrogen peroxide treatment pack 1 when needed. Creation of pre-fill weld 6 and post-fill weld 7 is most commonly accomplished through the application of heat and pressure at the desired location of the weld seam, thus the polyethylene layers of the adjoining laminated sheets melt and merge together in a chemically inert and air tight bond.

Foil pouch packaging of the general type indicated by foil pouch 2 are well known in the packaging industry and pre-fabricated foil pouches equipped with first opening slit 4 and/or second opening slit 5, pre-fill weld 6, and printed product information may be readily purchased from vendors specializing in foil pouch packaging for as little as $0.11 USD each in quantities of 30,000 or more. One such vendor is LPS Industries located at 10 Ceasar Place, Moonachie N.J. 07074 USA. In a preferred embodiment first laminated sheet 8 and second laminated sheet 9 consist of layered polyethylene, aluminum foil, and polyester with the polyethylene of both laminated sheets oriented toward each other, thus forming the internal lining of foil pouch 2. The middle foil layer of the laminated sheets forms a high performing vapor and gas barrier, and the outer polyester layer provides the necessary toughness and puncture resistance to prevent accidental opening or rupture of sealed hydrogen peroxide treatment pack 1. Instructions for opening the sealed pouch, mixing the solution, and applying the resulting solution to a wound are printed on the pouch for easy and immediate reference when needed. Although not deemed to be needed in most circumstances, additional toughness and puncture resistance can be obtained by using laminated sheets that also include a layer of nylon between the foil and polyethylene layers.

In a preferred embodiment percarbamide (CAS Number 124-43-6) is used as the hydrogen peroxide adduct 3. Percarbamide has a solubility sufficient to create a hydrogen peroxide concentration of 3% or higher, the complementary component, urea, is already present in the body and is non-toxic, percarbamide is commercially available, relatively inexpensive, and is relatively stable for the temperatures above 50° C. 20.75 grams of percarbamide are used to fill a nominal four inch wide by four inch long hydrogen peroxide treatment pack 1 sufficiently to allow the mixing and creation of a 250 ml hydrogen peroxide solution of equivalent concentration and strength as the USP standard for topical hydrogen peroxide (3%). Unopened hydrogen peroxide treatment pack 1 package weighs about 25 grams and is easily transportable, representing a greater than 10:1 weight difference as compared to a standard hydrogen peroxide solution of the same resulting volume.

One skilled in the art can appreciate that any number of different packaging configurations could be used without departing from the nature of the invention. Other possible packaging configuration include, but are not limited to: blister packaging, bottle and cap, LDPE blow filled tubes, and sealed metal canisters.

### Example

A treatment package was manufactured in accordance with the teachings and preferred embodiment described previously of the present invention. One adult and two minors backpacked in Sierra Nevada Mountain Range on the edge of Desolation Wilderness in California. The normal trail being inundated with water from spring snowmelt run-off the group decided to take an alternative route that resulted travelling through Manzanita brush. The result was that all three subjects received multiple wounds to both exposed legs. The wounds ranged from minor scratches to one significant freely bleeding gash. The instructions for mixing the treatment package with available water were followed resulting in a 250 ml treatment solution that was used to wash and irrigate the wounds on the left leg of all three subjects, which, since the freely bleeding gash was on the left leg of one of the subjects, included treating the freely bleeding gash. The wounds on the right legs of the subject were cleaned with the same available water that was used to make the treatment solution. Upon application of the 250 ml treatment solution, there was evident bubbling consistent with the application of hydrogen peroxide. All three subjects reported feeling more stinging, consistent with the presence of hydrogen peroxide, when treated with the 250 ml treatment solution. After 24 hours the wounds of the left and right legs of all three subjects were compared. In each case, the treated wounds of the left legs were less inflamed, less painful to touch, had more established scabbing, and visually appeared to have progressed more rapidly towards healing than the wounds of the right legs. The most significant wound, the freely bleeding gash, stopped bleeding soon after treatment and progressed toward healing in what appeared to be an expedited manner as compared to the less significant wounds on the right leg of the same subject.

Although the description above contains many specificities, these should not be construed as limiting the scope of the invention but as merely providing illustrations of some of the presently preferred embodiments of this invention. Thus the scope of this invention should be determined by the appended claims and their legal equivalents.

What is claimed is:

1. A treatment pack, comprising:
b. A sealed package;
c. Said sealed package containing an adduct of hydrogen peroxide;
d. Said packaging providing a barrier against contamination from moisture prior to packaging being opened;
e. Said package having means to be opened when desired;
f. Said package containing sufficient adduct of hydrogen peroxide when mixed with water produces an aqueous solution containing hydrogen peroxide.

2. A treatment pack as in claim 1, wherein when said adduct of hydrogen peroxide has sufficient solubility that when a pre-measured amount of said adduct of hydrogen peroxide is mixed with a pre-determined volume of said water an aqueous solution of at least 3% hydrogen peroxide is produced.

3. A method of treating wounds consisting of:
a. Sealing an adduct of hydrogen peroxide in a package with moisture barrier properties
b. Opening said package and mixing contents with water
c. Applying resulting solution to wound

4. A treatment as in claim 3, wherein said package contains a specified amount of said adduct of hydrogen peroxide

5. A treatment as in claim 3, wherein said package includes instructions that specify a pre-determined amount of water for mixing with said adduct of hydrogen peroxide.

6. A treatment pack consisting of:
a. A sealed package
b. Instructions
c. An adduct of hydrogen peroxide

7. A treatment pack as in claim 6, wherein said adduct of hydrogen peroxide is contained in said sealed package.

8. A treatment pack as in claim 6, wherein said instructions are printed on said sealed package.

9. A treatment pack as in claim 6, wherein said instructions are included with but external to said sealed package.

10. A treatment pack as in claim 6, wherein said instructions are included inside said sealed package.

11. A treatment pack as in claim 6, wherein the instructions specify mixing the said adduct of hydrogen peroxide with water

12. A treatment pack as in claim 6, wherein the instructions specify mixing the said adduct of hydrogen peroxide with a specified amount of water.

13. A treatment pack as in claim 6, wherein the instructions specify mixing said adduct of hydrogen peroxide with water and applying resulting solution to a wound
